(19) United States
(12) Patent Application Publication
(10) Pub. No.: US 2003/0092683 A1
(43) Pub. Date: May 15, 2003

(54) Use of tetracyclines as neuro-protective agents and for the treatment of parkinson's disease and related disorders

(76) Inventors: Yansheng Du, Carmel (IN); Richard Dodel, Marburg (DE)

Correspondence Address: RATNERPRESTIA, P O BOX 980, VALLEY FORGE, PA, 19482-0980, US

(21) Appl. No.: 10/007,850

(22) Filed: Nov. 13, 2001

(51) Int. Cl. A61K 31/65

(52) U.S. Cl. 514/152

(57) ABSTRACT

As discussed above the present invention in a first aspect thereof relates to the use of a tetracycline, preferably minocycline, for manufacture of a pharmaceutical composition for treatment or prevention of a disorder selected from Parkinson's disease and related disorders, preferably Parkinson's disease.

In a second aspect thereof the invention relates to a method for treating or prevention of a disorder selected from Parkinson's disease and related disorders in a patient in need thereof, which method includes administering the patient an effective dosage of a tetracycline, preferably minocycline.

In a third aspect thereof the invention relates to the use of a tetracycline, preferably minocycline, as a neuro-protective agent in general.

### BACKGROUND OF THE INVENTION

Parkinson's disease is a common and disabling neuro-degenerative disorder resulting from the depletion of brain dopamine caused by the dramatic loss of dopaminergic neurons in the substantia nigra pars compacta. The disease is one of the most common neurodegenerative diseases in elderly humans with an occurrence of about 1% of all over 60 years of age. The disease thus causes considerable concerns especially in the western hemisphere.

Therapies for Parkinson's disease currently available include dopamine-substitution with levodopa optionally in combination with dopamine agonists. These therapies currently available for Parkinson's disease help alleviate symptoms, however, efficacy is lost over time since progressive loss of dopaminergic neurons continues as the disease progresses. One of the most desirable therapeutic goals for both the treatment as well as prevention of Parkinson's disease, which would be neuro-protection, has been relatively elusive. Accordingly, there are no therapeutic efficacious neuro-protective drugs available to treat Parkinson's disease.

Testing of any drugs for treatment and/or prevention of Parkinson's disease is typically carried out in an animal model. In this model Parkinson is induced by administering 1-methyl-4-phenyl-1,2,3,6-tetrahydropyridine (MPTP), a neurotoxin. MPTP is metabolized into 1-methyl-4-phenylpyridinium (MPP⁺) which then selectively kills dopaminergic neurons thereby inducing Parkinsonism. The striking pathologic and clinical similarities between idiopathic Parkinson's disease and MPTP-induced Parkinsonism suggest that the two disorders share common pathogenic mechanism. MPTP/MPP+-induced neurodegeneration of dopaminergic neurons is a well characterized model which is therefore widely used to understand the pathogenesis of Parkinson's disease in a variety of primate and rodent models.

### SUMMARY OF THE INVENTION

These and other objects are achieved by the method and uses of the invention. The invention thus relates in a first aspect thereof to the use of a tetracycline for manufacture of a pharmaceutical composition for treatment or prevention of a disorder selected from Parkinson's disease and related disorders.

In a second aspect thereof the invention relates to a method for treating or prevention of a disorder selected from Parkinson's disease and related disorders in a patient in need thereof, which method includes administering the patient an effective dosage of a tetracycline.

In a third aspect thereof the invention relates to the use of a tetracycline as a neuro-protective agent in general.

According to all aspects of the invention in one embodiment the tetracycline may be incorporated in a pharmaceutical preparation which is preferably for oral administration. In this case the pharmaceutical preparation is even more preferably in a form selected from the group consisting of tablets, dragees, capsules, microtablets, pellets, a granulate, sirup, liquid and powder. According to another embodiment the pharmaceutical preparation may be for injection. Oral administration is preferred.

The Parkinson related disorder to be treated or prevented according to all aspects of the invention is preferably selected from the group consisting of multi-system atropy, progressive supranuclear body disease, Lewy body disease, and Alzheimer's disease.

### BRIEF DESCRIPTION OF THE DRAWINGS

FIG. 1 is a bar diagram showing effects of treatment with minocycline.

### DETAILED DESCRIPTION OF THE INVENTION

Tetracyclines are a class of antibiotics produced by various streptomyces species. Chemically the tetracyclines have a naphthacene skeleton i.e. are polycyclic hydrocarbons. Tetracyclines are known to have bacteriostatic and/or bacteriotoxic effects on a variety of gram positive and gram negative bacteria and are thus commonly used to treat bacterial infections.

The tetracyclines include natural tetracyclines of the first generation such as tetracycline, aureomycine, terramycine, demethylchlortetracycline, and semisynthetic tetracyclines of the second generation such as rolitetracycline, methacycline, doxycycline and minocycline.

Minocycline is further known to exert anti-inflammatory effects that are completely separate and distinct from its antimicrobial action. Clinical studies have shown that minocycline, and related tetracyclines, have beneficial anti-inflammatory activity and appear to be useful for treating both rheumatoid arthritis and osteoarthritis. Recently, minocycline has also been reported to be effective in models of both ischemia and Huntington disease (Yrjanheikki J et al. Proc Natl Acad Sci USA 96: 13496-13500; Chen M. et al. Nat Med 6: 797-801).

In view of the above it is thus a goal of the present invention to provide a new method of treating Parkinson's disease and related disorders which new therapy is effective over time.

It is another goal of the invention to provide a method of neuro-protection and suitable pharmaceutical compositions therefore.

It is another goal of the present invention to provide a new method of preventing Parkinson's disease and related disorders.

As discussed above the present invention in a first aspect thereof relates to the use of a tetracycline for manufacture of a pharmaceutical composition for treatment or prevention of a disorder selected from Parkinson's disease and related disorders, preferably Parkinson's disease.

The tetracyclines suitable for use in the present invention include, but are not limited to for example natural tetracyclines of the first generation such as tetracycline, aureomycine, terramycine, demethylchlortetracycline, and semisynthetic tetracyclines of the second generation such as rolitetracycline, methacycline, doxycycline and minocycline and their pharmaceutically acceptable salts. Most preferably the tetracycline is minocycline.

Suitable salts of the tetracyclines are e.g. acid addition salts for example hydrogen chlorides. Of course mixtures of tetracyclines and/or tetracycline salts may be used.

In a second aspect thereof the invention relates to a method for treating or prevention of a disorder selected from Parkinson's disease and related disorders in a patient in need thereof, which method includes administering the patient an effective dosage of a tetracycline, preferably a tetracycline as defined above.

In a third aspect thereof the invention relates to the use of a tetracycline, preferably minocycline, as a neuro-protective agent in general.

According to all aspects of the invention the tetracycline may be incorporated in a pharmaceutical composition. In one embodiment a pharmaceutical preparation is preferably for oral administration. In this case the pharmaceutical preparation is even more preferably in a form selected from the group consisting of tablets, dragees, capsules, microtablets, pellets, a granulate, sirup, liquid and powder. According to another embodiment the pharmaceutical preparation may be for injection. Oral administration and hence preparations suitable therefore are preferred.

In any case the pharmaceutical composition may comprise the tetracycline in combination with a pharmaceutically acceptable carrier or excipients. Optionally the composition may further comprise additional active ingredients.

The effective dosage of the tetracycline to be administered will depend on the tetracycline itself, the condition of the patient to be treated and/or whether administration is for actual treatment or prevention. Suitable dosages can be determined by the skilled medical practitioner by routine experiments. In general the dosages to be administered will be the same or higher than typical tetracycline dosages used in antibacterial treatments.

The Parkinson related disorder to be treated or prevented according to all aspects of the invention is preferably selected from the group consisting of multi-system atropy, progressive supranuclear body disease, Lewy body disease, and Alzheimer's disease. Preferably the disorder is Parkinson.

To investigate the neuroprotective effects of tetracyclines minocycline was used. The compound was tested on MPTP-induced neuronal death in vivo as shown in the following examples which are given for illustration purposes only and are not to be construed as limiting the invention. It was also tested neuroprotective in 6-hydroxy-and NO-mediated cell death in neuronal cell cultures.

### EXAMPLES

### Example 1

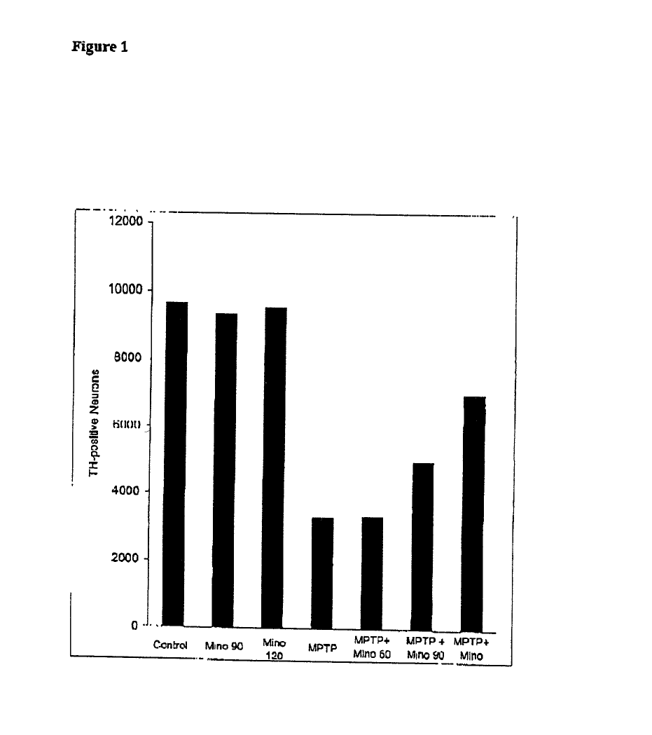

In a first test C57BL/6 mice were dosed with 60, 90, and 120 mg/kg minocycline p.o. daily for nine days. On day 3 mice were injected with MPTP (20 mg/kg×4). Seven days following the last injection of MPTP, the brains were analyzed by immun histochemistry to detect and quantify tyrosin hydroxylase (TH)-positive neurons. Mice treated with MPTP alone showed a 63% reduction in TH-positive neurons. A significant neuro-protective effect was observed in the MPTP-injected mice which are pretreated with minocycline at 90 and 120 mg/kg. Mice that received daily treatments of minocycline at either 90 or 120 mg/kg and MPTP showed increased viable TH-positive neurons in the SNpc, ranging from 37% of control (no minocycline treatment) to 56% (90 mg/kg) and 77% (120 mg/kg) of control following minocycline treatment. The results are shown in FIG. 1.

Referring to FIG. 1 Dopaminergic neurons are resistant to MPTP neurotoxicity after treatment with minocycline. TH immuno-staining of a representative midbrain section 7 days after MPTP treatment (i.p. 4 injection of 20 mg/kg) in the absence or presence of minocycline (60, 90, 120 mg/kg daily). Left to right bars: saline (control), minocycline at 90 and 120 mg/kg alone, MPTP treated, MPTP treated after 2 hr pretreatment of minocycline 60 mg/kg. MPTP treated after 2 hr pretreatment of minocycline 90 mg/kg, MPTP treated after 2 hr pretreatment of minocycline 120 mg/kg. Minocycline at 90 and 120 mg/kg significantly protects TH-positive neurons following MPTP exposure.

### Example 2

To confirm that the effects of minocycline did not result from modifying the conversion rate of MPTP to MPP+, the activity of monoamine oxidase B (MAO B), an enzyme responsible for the conversion of MPTP to MPP+, as well as actual MPP+ levels in the midbrain were measured in the absence or presence of minocycline.

The neuro-protective effect of minocycline was dose-dependent as the 60 mg/kg dose of minocycline failed to protect dopamine neurons from MPTP toxicity (FIG. 1). Minocycline alone did not alter the number of TH-positive neurons significantly.

### Example 3

Next the animals were treated with minocycline (120 mg/kg, p.o.) 4 h and 24 h after MPTP administration. Interestingly, minocycline treatment significantly protects against MPTP-induced dopamine neurotoxicity even 4 h after the last (or 12 h after the first) MPTP injection. Mice that received minocycline beginning 4 h following MPTP treatment showed increased viable TH-positive neurons in the SNpc, ranging from 36% of control (no minocycline treatment) to 66% of control following minocycline (120 mg/kg) treatment (p<0.05).

Having thus described our invention with reference to certain specific embodiments, the present invention is nevertheless not limited to the details shown. Rather, various modification may be made to the details within the scope and range of equivalents of the claims without departing from the spirit of the invention.

What is claimed:

1. Use of a tetracycline for manufacture of a pharmaceutical composition for treatment or prevention of a disorder selected from Parkinson's disease and related disorders.

2. The use according to claim 1, wherein the tetracycline is selected from the group consisting of tetracycline, aureomycine, terramycine, demethylchlortetracycline, rolitetracycline, methacycline, doxycycline and minocycline and their pharmaceutically acceptable salts.

3. The use according to claim 2, wherein the tetracycline is minocycline.

4. The use according to claim 1, wherein the pharmaceutical preparation is for oral administration.

5. The use according to claim 4, wherein the pharmaceutical preparation is in a form selected from the group consisting of tablets, dragees, capsules, microtablets, pellets, a granulate, sirup, liquid and powder.

6. The use according to claim 1, wherein the pharmaceutical preparation is for injection.

7. Use of minocycline for preparation of a pharmaceutical composition for treatment or prevention of a disorder selected from Parkinson's disease and related disorders, wherein the pharmaceutical preparation is for oral administration.

8. A method for treating or prevention of a disorder selected from Parkinson's disease and related disorders in a patient in need thereof, which method includes administering the patient an effective dosage of a tetracycline.

9. The method according to claim 8, wherein the tetracycline is selected from the group consisting of tetracycline, aureomycine, terramycine, demethylchlortetracycline, rolitetracycline, methacycline, doxycycline and minocycline and their pharmaceutically acceptable salts.

10. The method of claim 9, wherein the tetracycline is minocycline.

11. The method according to claim 8, wherein the tetracycline is administered orally.

12. The method according to claim 11, wherein the pharmaceutical preparation is in a form selected from the group consisting of tablets, dragees, capsules, micro-tablets, pellets, a granulate, sirup, liquid and powder.

13. The method of claim 8, wherein the tetracycline is administered by injection.

14. A method for treating or prevention of a disorder selected from Parkinson's disease and related disorders in a patient in need thereof, which method includes orally administering the patient an effective dosage of minocycline.

15. The use of claim 1, wherein the Parkinson related disorder is selected from the group consisting of multi-system atropy, progressive supranuclear body disease, Lewy body disease, and Alzheimer's disease.

16. The method of claim 8, wherein the Parkinson related disorder is selected from the group consisting of multi-system atropy, progressive supranuclear body disease, Lewy body disease, and Alzheimer's disease.

17. Use of a tetracycline as a neuro-protective agent.

18. The use according to claim 17, wherein the tetracycline is selected from the group consisting of tetracycline, aureomycine, terramycine, demethylchlortetracycline, rolitetracycline, methacycline, doxycycline and minocycline and their pharmaceutically acceptable salts.

19. The use of claim 18, wherein the tetracycline is minocycline.
